(19) United States
(12) Patent Application Publication
(10) Pub. No.: US 2026/0253714 A1
(43) Pub. Date: Aug. 27, 2026

(54) AI-DRIVEN MEDICAL INTAKE ASSISTANT

(71) Applicant: Wynk Health, Inc., Georgetown, MA (US)

(72) Inventor: Matthew Racki, Georgetown, MA (US)

(21) Appl. No.: 19/548,457

(22) Filed: Feb. 24, 2026

Related U.S. Application Data
(60) Provisional application No. 63/762,841, filed on Feb. 25, 2025.

(51) Int. Cl. G16H 40/20; A61B 5/00; G16H 10/20; G16H 10/60

(52) U.S. Cl. CPC G16H 40/20; A61B 5/0022; A61B 5/4803; A61B 5/7264; A61B 5/7282; A61B 5/746; G16H 10/20; G16H 10/60

(57) ABSTRACT

An AI-driven medical intake assistant system includes a computing device having a processor and memory configured to execute an intake module, an abnormality detection module, and an electronic health record (EHR) integration module. The intake module presents a conversational interface that dynamically selects patient intake questions using a rule-based branching decision tree with clinical topic tags and transition rules. One or more connected diagnostic devices automatically capture patient vital sign measurements, including blood pressure, temperature, pulse oximetry, or weight, and transmit the measurements to the computing device. The abnormality detection module compares captured measurements to predefined threshold values or historical baseline data retrieved from the EHR and generates provider-facing alerts when deviation thresholds are exceeded. The EHR integration module maps patient responses and vital sign measurements into predefined structured EHR fields prior to a provider consultation.

### CROSS REFERENCE TO RELATED APPLICATIONS

This Application claims the benefit of U.S. Provisional Patent Application No. 63/762,841, filed February 25, 2025, the contents of which are incorporated herein by reference in its entirety as if fully set forth.

### FIELD

Illustrative embodiments relate to healthcare automation and clinical workflow optimization, and more particularly, various embodiments relate to an artificial intelligence (AI)-powered medical assistant that automates patient intake, documentation, and education.

### BACKGROUND

Patient intake and documentation processes sometimes rely on manual data collection, vitals measurement, and documentation, which are often time-consuming and inefficient. In most clinical environments, a nurse or medical assistant must escort a patient to an exam room, collect medical history, manually measure vitals, and transcribe findings into the electronic health record (EHR). This workflow introduces several inefficiencies, including administrative burden and physician burnout. Studies indicate that physicians spend nearly 49% of their day on EHR tasks, including documentation, data entry, and note-taking. This manual documentation reduces patient face-to-face time, impacting the quality of care and increasing physician burnout. Additionally, data entry errors and inconsistent documentation are common, as transcription errors occur when patient-reported data is manually recorded by medical staff. A lack of standardization in documentation results in inconsistent patient records, making it harder for clinicians to make informed decisions.

### SUMMARY OF VARIOUS EMBODIMENTS

An intelligent medical assistant is introduced that automates patient intake, provides real-time scribing and documentation support, and enhances patient education during provider interactions. The system significantly reduces administrative workload, improves accuracy, and fosters a more interactive, informed patient experience. By integrating AI, connected devices, EHR automation, and contextual education, this system represents a novel advancement in medical workflow efficiency, eliminating manual bottlenecks and improving overall clinical productivity.

In an embodiment, an AI-driven medical intake assistant system includes a computing device with a processor and non-transitory memory executing an intake module that presents a conversational interface to a patient, receives patient responses, and dynamically selects follow-up intake questions using a rule-based branching decision tree with clinical topic tags and transition rules; one or more connected diagnostic devices that automatically capture and transmit vital sign measurements including blood pressure, temperature, pulse oximetry, or weight; an abnormality detection module that compares captured measurements to predefined threshold values or historical patient baseline data from an electronic health record (EHR) and generates a provider-facing alert when a predefined deviation threshold is exceeded; and an EHR integration module that maps the patient responses and vital sign measurements into predefined structured EHR fields, including at least a History of Present Illness (HPI) or Review of Systems (ROS) section, prior to a provider consultation.

In some embodiments, the intake module is executed using either an on-premises computing device within a healthcare provider network or a remote cloud-based large language model accessed via an encrypted communication channel.

In some embodiments, the system further includes a scribe module that receives audio of a provider-patient interaction, converts the audio into text using speech recognition, extracts medical entities from the text using natural language processing, and generates a structured clinical note including a SOAP-format document for provider review prior to entry into the EHR.

In some embodiments, the system further includes a clinical decision support module that applies stored clinical rules to extracted medical entities and captured vital sign measurements and generates provider-reviewable recommendations—including diagnostic tests, laboratory orders, or treatment considerations—when predefined rule conditions are satisfied.

In some embodiments, the abnormality detection module calculates a deviation metric between a current vital sign measurement and historical measurements retrieved from the EHR using at least one of a predefined absolute threshold, a rolling average comparison, an exponentially weighted moving average, or a regression-based trend analysis, and generates an alert when the deviation metric exceeds a stored threshold value.

In some embodiments, the system further includes a contextual patient education module that identifies a diagnosis or treatment topic from extracted medical entities, queries an education content library storing assets tagged by condition and language, and transmits selected educational content to an in-room display device, a patient portal, or a mobile device associated with the patient.

In an embodiment, a computer-implemented method for AI-driven patient intake and documentation includes presenting intake questions via a conversational interface selected using a stored rule-based decision tree; receiving patient responses and storing structured intake data in memory; automatically receiving vital sign measurements from one or more connected diagnostic devices; comparing the vital sign measurements to predefined threshold values or historical baseline data retrieved from an electronic health record; generating a provider alert when a deviation threshold is exceeded; mapping the structured intake data and vital sign measurements into predefined electronic health record fields; receiving audio of a provider-patient interaction; converting the audio to text and extracting medical entities; and generating a structured clinical note for provider review prior to storage in the electronic health record.

In some embodiments, at least a portion of the conversational interface or the clinical note generation is executed using either an on-premises computing device or a remote cloud-based large language model accessed through encrypted communication.

### BRIEF DESCRIPTION OF THE DRAWINGS

The following drawings illustrate various aspects of the AI-driven medical intake assistant and how it improves upon traditional patient intake workflows. Those skilled in the art should more fully appreciate advantages of various embodiments from the following “Description of Illustrative Embodiments,” discussed with reference to the drawings summarized immediately below.

FIG. 1 shows an example patient intake process in accordance with various embodiments.

FIG. 2 shows an example AI-Enhanced intake workflow in accordance with various embodiments.

FIG. 3 shows an example scribe mode workflow in accordance with various embodiments.

FIG. 4 shows an example contextual education feature in accordance with various embodiments.

FIG. 5 shows an example AI deployment architecture workflow in accordance with various embodiments.

FIG. 6 shows an example computing device in accordance with various embodiments.

### DETAILED DESCRIPTION

In various embodiments, the AI-driven medical intake assistant automates patient intake, real-time documentation, and patient education by integrating artificial intelligence, connected diagnostic devices, and electronic health record (EHR) automation into a single workflow. This system improves efficiency, reduces administrative burden, and enhances patient engagement by intelligently managing intake, transcription, and decision support during provider-patient interactions. This system integrates artificial intelligence, connected diagnostic devices, and real-time EHR automation to enhance clinical efficiency, reduce administrative burden, and improve patient engagement.

In some situations, inefficiencies include delayed and fragmented data integration, where vital signs are often measured separately from intake questioning, requiring staff to manually enter these values into the EHR after the fact. This delay can lead to incomplete or outdated patient information by the time the physician enters the exam room. Additionally, there is a lack of real-time clinical decision support, as current patient intake workflows do not provide intelligent alerts for potential health risks based on historical patient data. Physicians rely solely on their own review of prior records, which may overlook trends in vital signs or symptoms over time. Another limitation is limited patient education and engagement, as most patient education is limited to verbal explanations or static printed materials handed out after the visit. There is no standardized way to provide real-time, interactive educational materials that adapt to the provider’s conversation with the patient.

Various AI-powered healthcare solutions attempt to address individual components of the intake and documentation workflow but lack a fully integrated approach. Self-service kiosks automate intake but lack real-time provider assistance and decision support. Voice-based transcription solutions, capture spoken notes but do not integrate historical data analysis or AI-generated education. Digital check-in systems, such as Phreesia, streamline form-based intake but lack dynamic patient engagement, scribing, and decision support.

To overcome these limitations, there is a critical need for an AI-driven system that automates patient intake and documentation while seamlessly integrating structured data into the EHR. The ideal system would capture and analyze vital signs in real time using connected diagnostic devices and leverage AI-powered transcription (Scribe Mode) to document provider-patient conversations while suggesting clinical decisions. Additionally, it should identify trends in historical patient data to flag abnormal findings automatically and deliver interactive patient education during and after the visit, aligned with the provider’s discussion.

Described herein is an intelligent medical assistant that automates patient intake, provides real-time scribing and documentation support, and enhances patient education during provider interactions. The system significantly reduces administrative workload, improves accuracy, and fosters a more interactive, informed patient experience. By integrating AI, connected devices, EHR automation, and contextual education, this system represents a novel advancement in medical workflow efficiency, eliminating manual bottlenecks and improving overall clinical productivity.

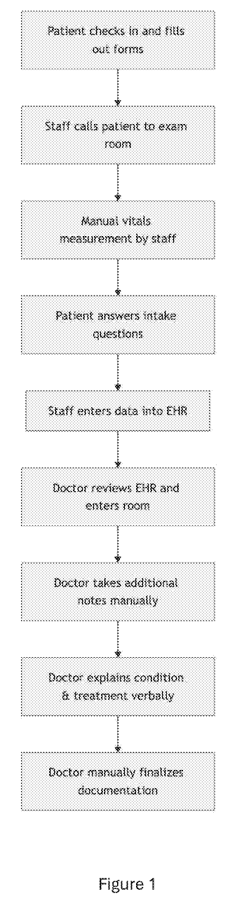

FIG. 1 shows an example Patient Intake Process in accordance with various embodiments. In various embodiments, FIG. 1 illustrates patient check-in and intake workflow, where a nurse or medical assistant manually collects patient history, measures vital signs, and transcribes information into the EHR. This process may be prone to inefficiencies, including transcription errors, delays in data entry, and increased administrative burden on clinical staff.

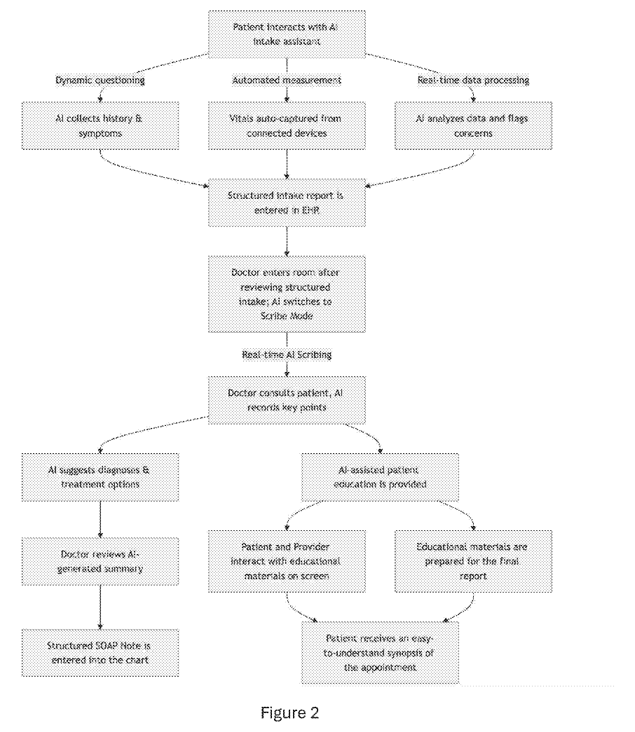

FIG. 2 shows an example AI-Enhanced Intake Workflow in accordance with various embodiments. In various embodiments, FIG. 2 demonstrates how the AI-driven assistant automates patient intake. The system engages the patient through a conversational AI interface, dynamically adjusting questions based on patient responses and retrieving relevant medical history from the EHR. Connected diagnostic devices automatically record vital signs and flag abnormal readings for provider review. The structured data is transmitted to the EHR in real-time, ensuring that the provider has complete and accurate information before entering the exam room.

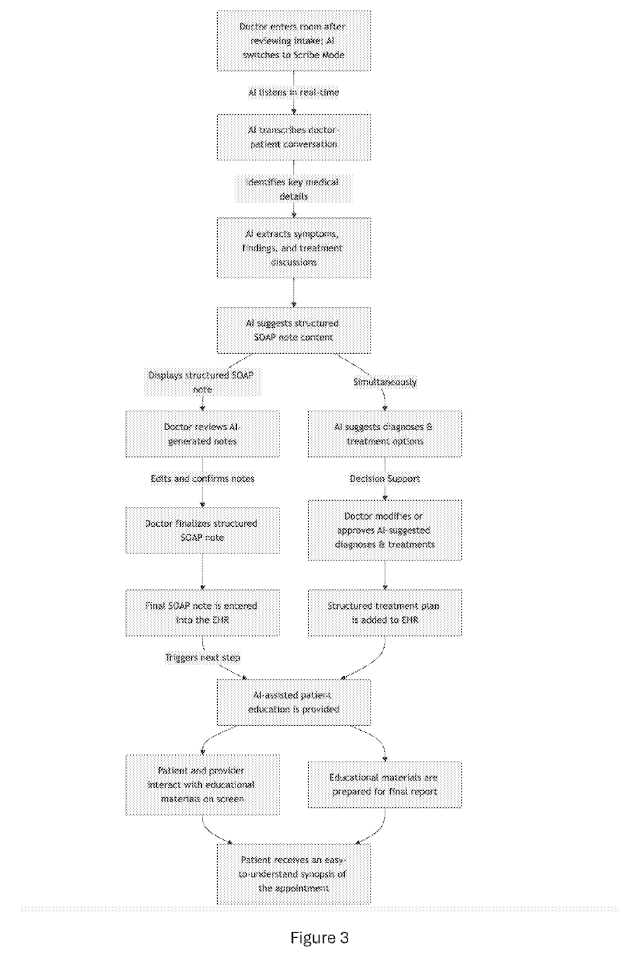

FIG. 3 shows an example Scribe Mode Workflow in accordance with various embodiments. In various embodiments, FIG. 3 illustrates the AI-powered transcription and documentation process during a provider-patient consultation. The system listens to the conversation, transcribes key medical details, and suggests potential diagnoses and treatments. The provider can review, modify, and approve the AI-generated documentation before it is entered into the EHR, reducing the time spent on manual note-taking and improving documentation accuracy.

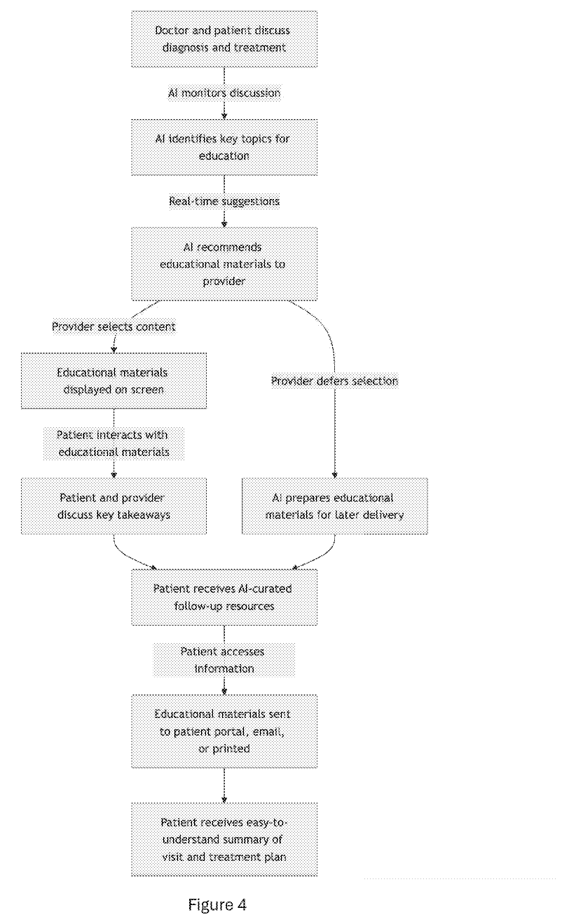

FIG. 4 shows an example contextual education feature in accordance with various embodiments. In various embodiments, FIG. 4 highlights how the system provides real-time, interactive educational materials tailored to the patient’s condition. As the provider discusses a diagnosis or treatment plan, the AI assistant suggests relevant educational content, which can be displayed on a tablet or kiosk in the exam room. Patients can engage with these materials during the visit and receive follow-up resources via email, the patient portal, or printed handouts, enhancing their understanding and compliance with the treatment plan.

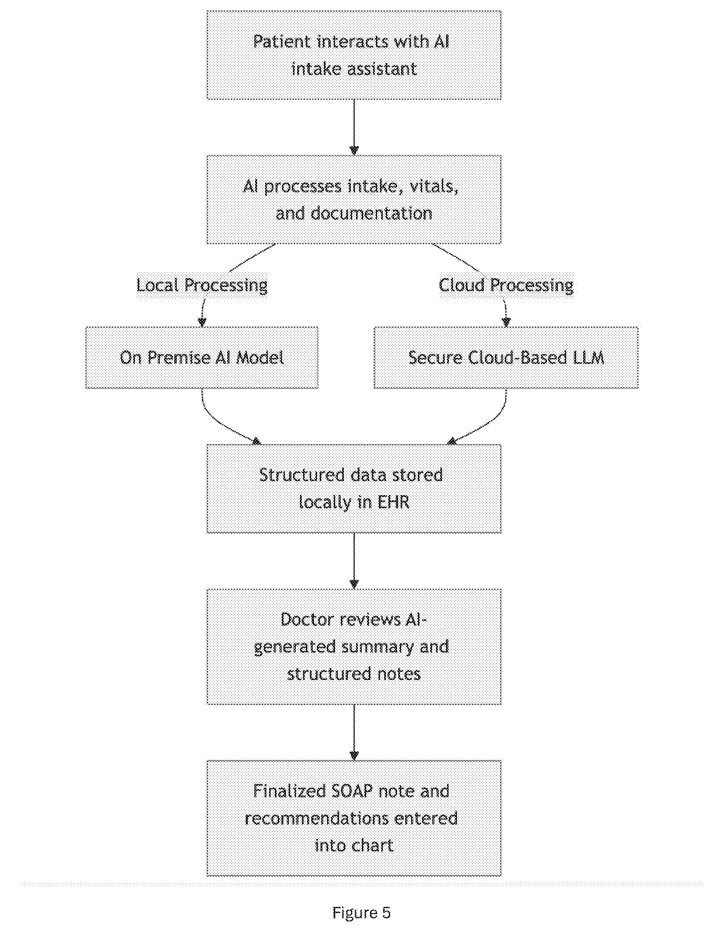

FIG. 5 shows an example AI deployment architecture workflow in accordance with various embodiments. In various embodiments, FIG. 5 illustrates the flexible deployment options for the AI-driven medical intake assistant, allowing healthcare organizations to choose between on-premises AI processing or cloud-based AI processing based on their regulatory, security, and operational needs. The AI intake assistant processes patient-reported symptoms, vital signs, and documentation, then determines whether to handle AI processing locally on a secure, on-premises server or via a HIPAA-compliant cloud-based LLM. If the on-premises AI model is selected, all AI processing occurs within the healthcare provider’s private network, ensuring that sensitive patient data never leaves the facility. This is ideal for organizations requiring strict data security and HIPAA compliance. If the cloud-based AI model is selected, patient data is securely transmitted to a remote HIPAA-compliant AI environment, where the LLM processes intake information, analyzes clinical insights, and generates structured documentation. Regardless of the chosen deployment, the AI system structures intake data, SOAP notes, and documentation, which is then recorded in the EHR for provider review before finalization. This architecture ensures that healthcare organizations can maintain control over data security while benefiting from AI-driven efficiency and automation

The AI-guided intake and dynamic questioning process begins when the intake module engages the patient through a conversational AI interface, which can be accessed via a tablet or voice-enabled device in the exam room. The AI dynamically adjusts its questioning based on the patient’s responses, ensuring that all relevant medical history and symptoms are captured before the provider enters the room. Unlike traditional intake, which relies on static forms or scripted questions, this adaptive AI-driven process ensures that no critical details are overlooked. Additionally, the system pulls historical medical data from the EHR to personalize the intake experience. For example, if a patient has a history of hypertension, the AI may ask follow-up questions about medication adherence, recent blood pressure trends, or lifestyle changes that could affect their condition.

In an embodiment, the adaptive questioning engine operates using a rule-based branching decision tree stored in memory. Each question node is associated with a clinical topic tag, triggering conditions, follow-up question sets, and termination criteria. For example, if a patient reports chest pain, the system activates a cardiovascular branch and sequentially prompts for onset, duration, severity, radiation, and associated symptoms. Logical rules may govern branch transitions.

The automated vital sign collection process allows connected diagnostic devices, such as digital blood pressure monitors, thermometers, pulse oximeters, and weight scales, to seamlessly integrate into the intake workflow. As the patient interacts with the AI assistant, the system prompts them to use these devices, which automatically capture and record vital sign measurements. The data is transmitted in real-time to the AI system and analyzed for abnormalities, comparing the current readings with historical patient records stored in the EHR. If a significant deviation is detected, such as a sudden increase in blood pressure compared to past visits, the system can flag the anomaly for provider review or suggest a retest to ensure accuracy.

In an embodiment, abnormality detection may use predefined clinical thresholds stored in memory. For example, systolic blood pressure greater than 140 mmHg, oxygen saturation below 92%, or temperature exceeding 100.4°F may trigger an alert.

In another embodiment, the system compares current readings with historical patient baselines retrieved from the EHR.

An absolute value threshold, or alternatively, rolling averages, exponentially weighted moving averages, or regression-based trend analyses may be used to detect progressive changes over multiple visits. If a significant deviation is detected, the system may prompt for retesting, notify clinical staff, or generate a provider-facing alert prior to the encounter.

The EHR integration and real-time data structuring ensure that all patient-reported symptoms, medical history, and vital sign measurements are directly entered into the appropriate sections of the patient’s EHR. Unlike traditional methods that require staff to manually transcribe intake forms and vital signs, this system automates the process, reducing transcription errors and ensuring data is available for provider review before the consultation begins. Additionally, the AI can structure the intake information into standardized clinical note formats, including the History of Present Illness (HPI) and Review of Systems (ROS) sections, improving the consistency and completeness of documentation.

In an embodiment, a structured mapping engine converts extracted entities into standardized clinical formats. For example: Symptom entities with time references are mapped to the History of Present Illness (HPI); Past diagnoses are mapped to Past Medical History (PMH); Objective measurements are mapped to the Objective section; and/or Review-of-systems findings are mapped to ROS fields.

The scribe mode with real-time transcription and decision support activates when the provider enters the room, allowing the AI to passively listen to the conversation and transcribe key clinical details. Using advanced speech recognition and natural language processing (NLP), the system identifies medically relevant information, including symptoms, diagnoses, and treatment discussions. The AI then organizes this data into structured clinical documentation, such as SOAP notes, which the provider can review and approve before finalizing the encounter. Additionally, the system provides real-time clinical decision support by suggesting possible diagnoses or treatment options based on the conversation. For example, if the provider and patient discuss persistent fatigue and unintentional weight loss, the AI may suggest evaluating thyroid function or screening for metabolic conditions. These suggestions appear discreetly on the provider’s screen, allowing them to accept, modify, or dismiss the recommendations without disrupting the patient interaction.

The contextual patient education feature enhances patient engagement by delivering real-time, interactive educational materials based on the ongoing discussion between the provider and patient. If the provider diagnoses the patient with hypertension, for example, the AI can automatically display educational content on managing high blood pressure, including lifestyle modifications, medication adherence, and potential complications. This content can be presented through an in-room kiosk, tablet, or sent digitally to the patient’s portal for later review. By integrating educational materials into the consultation, the system helps improve patient understanding and adherence to treatment plans.

In an embodiment, educational assets are stored in a content library and tagged by condition, severity, age group, literacy level, and language. When a diagnosis or treatment topic is identified through entity recognition, the system queries the education library using matching tags and patient attributes.

The AI deployment options provide flexibility for healthcare organizations to implement the system according to their privacy and infrastructure requirements. The AI assistant can operate using an on-premises large language model (LLM) for organizations that prioritize data security and compliance with regulations such as HIPAA. Alternatively, a secure cloud-based LLM can be used to leverage scalable processing power and continuous AI model updates. This flexibility ensures that the system can be adopted by a wide range of healthcare settings, from small clinics to large hospital networks.

By combining AI-guided intake, automated vital sign collection, real-time transcription, and contextual education, described herein is a medical assistant that optimizes clinical workflows, reduces documentation burdens, and improves patient engagement. This fully integrated system enhances the accuracy and efficiency of patient encounters while allowing providers to focus more on direct patient care.

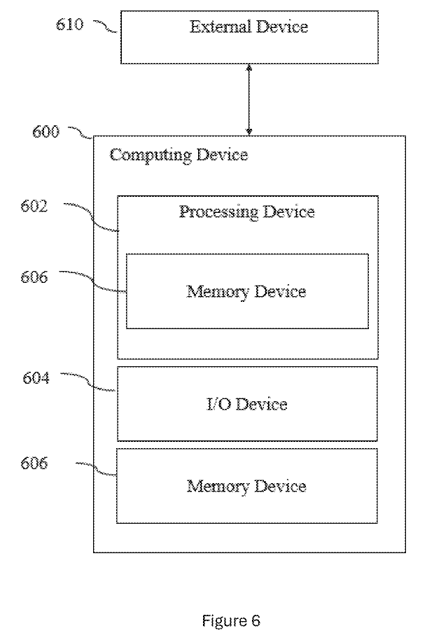

FIG. 6 shows an example computing device in accordance with various embodiments.

For example, FIG. 6 schematically shows a computing device 600 in accordance with various embodiments. The computing device 600 is one example of a computing device which is used to perform one or more operations of process/method illustrated above. The computing device 600 includes a processing device 602, an input/output device 604, and a memory device 606. The computing device 600 may be a stand-alone device, an embedded system, or a plurality of devices configured to perform the functions described above.

Furthermore, the computing device 600 may communicate with one or more external devices 610.

The input/output device 604 enables the computing device 600 to communicate with an external device 610. For example, the input/output device 604 may be a network adapter, a network credential, an interface, or a port (e.g., a USB port, serial port, parallel port, an analog port, a digital port, VGA, DVI, HDMI, FireWire, CAT 5, Ethernet, fiber, or any other type of port or interface), among other things. The input/output device 604 may be comprised of hardware, software, or firmware. The input/output device 604 may have more than one of these adapters, credentials, interfaces, or ports, such as a first port for receiving data and a second port for transmitting data, among other things.

The external device 610 may be any type of device that allows data to be input or output from the computing device 600. For example, the external device 610 may be a meter, a control system, a sensor, a mobile device, a reader device, equipment, a handheld computer, a diagnostic tool, a controller, a computer, a server, a printer, a display, a visual indicator, a keyboard, a mouse, or a touch screen display, among other things. Furthermore, the external device 610 may be integrated into the computing device 600. More than one external device may be in communication with the computing device 600.

The processing device 602 may be a programmable type, a dedicated, hardwired state machine, or a combination thereof. The processing device 602 may further include multiple processors, Arithmetic-Logic Units (ALUs), Central Processing Units (CPUs), Digital Signal Processors (DSPs), or Field-programmable Gate Arrays (FPGA), among other things. For forms of the processing device 602 with multiple processing units, distributed, pipelined, or parallel processing may be used. The processing device 602 may be dedicated to performance of just the operations described herein or may be used in one or more additional applications. The processing device 602 may be of a programmable variety that executes processes and processes data in accordance with programming instructions (such as software or firmware) stored in the memory device 606. Alternatively or additionally, programming instructions are at least partially defined by hardwired logic or other hardware. The processing device 602 may be comprised of one or more components of any type suitable to process the signals received from the input/output device 604 or elsewhere, and provide desired output signals. Such components may include digital circuitry, analog circuitry, or a combination thereof.

The memory device 606 in different embodiments may be of one or more types, such as a solid-state variety, electromagnetic variety, optical variety, or a combination of these forms, to name but a few examples. Furthermore, the memory device 606 may be volatile, nonvolatile, transitory, non-transitory or a combination of these types, and some or all of the memory device 606 may be of a portable variety, such as a disk, tape, memory stick, or cartridge, to name but a few examples. In addition, the memory device 606 may store data which is manipulated by the processing device 602, such as data representative of signals received from or sent to the input/output device 604 in addition to or in lieu of storing programming instructions, among other things. As shown in FIG. 6, the memory device 606 may be included with the processing device 602 or coupled to the processing device 602, but need not be included with both.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

While the present disclosure has been illustrated and described in detail in the drawings and foregoing description, the same is to be considered as illustrative and not restrictive in character, it being understood that only certain exemplary embodiments have been shown and described, and that all changes and modifications that come within the spirit of the present disclosure are desired to be protected. It should be understood that while the use of words such as “preferable,” “preferably,” “preferred” or “more preferred” utilized in the description above indicate that the feature so described may be more desirable, it nonetheless may not be necessary, and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. The term “of” may connote an association with, or a connection to, another item, as well as a belonging to, or a connection with, the other item as informed by the context in which it is used. The terms “coupled to,” “coupled with” and the like include indirect connection and coupling, and further include but do not require a direct coupling or connection unless expressly indicated to the contrary. When the language “at least a portion” or “a portion” is used, the item can include a portion or the entire item unless specifically stated to the contrary. Unless stated explicitly to the contrary, the terms “or” and “and/or” in a list of two or more list items may connote an individual list item, or a combination of list items. Unless stated explicitly to the contrary, the transitional term “having” is open-ended terminology, bearing the same meaning as the transitional term “comprising”.

Various embodiments described herein may be implemented at least in part in any conventional computer programming language. For example, some embodiments may be implemented in a procedural programming language (e.g., “C”), or in an object oriented programming language (e.g., “C++”). Other embodiments described herein may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments described herein may be implemented as a combination of both software (e.g., a computer program product) and hardware. Still other embodiments described herein are implemented as entirely hardware, or entirely software.

The embodiments described herein described above are intended to be merely exemplary; numerous variations and modifications will be apparent to those skilled in the art. Such variations and modifications are intended to be within the scope of the present application as defined by any of the appended claims. It shall nevertheless be understood that no limitation of the scope of the present disclosure is hereby created, and that the present disclosure includes and protects such alterations, modifications, and further applications of the exemplary embodiments as would occur to one skilled in the art with the benefit of the present disclosure.

1. An AI-driven medical intake assistant system, comprising:
a computing device including a processor and a non-transitory memory storing executable instructions;
an intake module executed by the processor and configured to:
present a conversational interface to a patient via a user device,
receive patient responses, and
dynamically select subsequent intake questions using a rule-based branching decision tree stored in memory, the decision tree including clinical topic tags and transition rules;
one or more connected diagnostic devices in communication with the computing device and configured to automatically capture patient vital sign measurements including blood pressure, temperature, pulse oximetry, or weight, and transmit the measurements to the computing device;
an abnormality detection module executed by the processor and configured to:
compare captured vital sign measurements to (i) predefined threshold values stored in memory or (ii) historical patient baseline data retrieved from an electronic health record (EHR), and
generate a provider-facing alert when the measurements exceed a predefined deviation threshold; and
an EHR integration module executed by the processor and configured to map patient responses and captured vital sign measurements into predefined structured fields of the EHR, including at least a History of Present Illness (HPI) or Review of Systems (ROS) section, prior to a provider consultation.

2. The system of claim 1, wherein the intake module is executed using either:
(a) an on-premises computing device located within a healthcare provider network, or
(b) a remote cloud-based large language model accessed via an encrypted communication channel.

3. The system of claim 1, further comprising a scribe module executed by the processor and configured to:
receive audio of a provider-patient interaction,
convert the audio into text using speech recognition,
extract medical entities from the text using natural language processing, and
generate a structured clinical note including a SOAP format document for provider review prior to entry into the EHR.

4. The system of claim 3, further comprising a clinical decision support module configured to:
apply stored clinical rules to extracted medical entities and captured vital sign measurements; and
generate provider-reviewable recommendations including diagnostic tests, laboratory orders, or treatment considerations when predefined rule conditions are satisfied.

5. The system of claim 1, wherein the abnormality detection module is further configured to:
calculate a deviation metric between a current vital sign measurement and historical measurements retrieved from the EHR using at least one of:
(a) a predefined absolute threshold,
(b) a rolling average comparison,
(c) an exponentially weighted moving average, or
(d) a regression-based trend analysis; and
generate an alert when the deviation metric exceeds a stored threshold value.

6. The system of claim 3, further comprising a contextual patient education module configured to:
identify a diagnosis or treatment topic from extracted medical entities;
query an education content library storing assets tagged by condition and language; and
transmit selected educational content to at least one of: an in-room display device, a patient portal, or a mobile device associated with the patient.

7. A computer-implemented method for AI-driven patient intake and documentation performed by a computing device including a processor and memory, comprising:
presenting, via a conversational interface, intake questions selected using a stored rule-based decision tree;
receiving patient responses and storing structured intake data in memory;
automatically receiving vital sign measurements from one or more connected diagnostic devices;
comparing the vital sign measurements to predefined threshold values or historical baseline data retrieved from an electronic health record;
generating a provider alert when a deviation threshold is exceeded;
mapping the structured intake data and vital sign measurements into predefined electronic health record fields;
receiving audio of a provider-patient interaction;
converting the audio to text and extracting medical entities; and
generating a structured clinical note for provider review prior to storage in the electronic health record.

8. The method of claim 7, wherein at least a portion of the conversational interface or clinical note generation is executed using either an on-premises computing device or a remote cloud-based large language model accessed through encrypted communication.
